(19) United States
(12) Patent Application Publication
(10) Pub. No.: US 2023/0240519 A1
(43) Pub. Date: Aug. 3, 2023

(54) METHOD FOR CALIBRATING ENDOSCOPE AND ENDOSCOPE SYSTEM

(71) Applicant: OLYMPUS MEDICAL SYSTEMS CORP., Tokyo (JP)

(72) Inventors: Katsuhiko YOSHIMURA, Koganei-shi (JP); Kosuke KISHI, Mitaka-shi (JP); Shun INOUE, Kokubunji-shi (JP); Noriaki YAMANAKA, Hachioji-shi (JP); Shota SAWADA, Kokubunji-shi (JP); Yuto MAEKAWA, Tokyo (JP); Shu KAMBE, Hachioji-shi (JP)

(73) Assignee: OLYMPUS MEDICAL SYSTEMS CORP., Tokyo (JP)

(21) Appl. No.: 18/132,541

(22) Filed: Apr. 10, 2023

Related U.S. Application Data
(63) Continuation-in-part of application No. 17/501,194, filed on Oct. 14, 2021, now abandoned.

(51) Int. Cl. A61B 1/005

(52) U.S. Cl. CPC A61B 1/0051; A61B 1/05

(57) ABSTRACT

A method for calibrating an endoscope, the endoscope including a curved portion that can be driven to bend and a flexible portion through which a bending wire that bends the curved portion is inserted, includes: arranging the curved portion and the flexible portion of the endoscope so that they have a predetermined shape; bending the curved portion; measuring first shape information, which is information about a shape of the curved portion; and updating a control parameter for the endoscope to drive the curved portion based on the measured first shape information.

### CROSS-REFERENCE TO RELATED APPLICATIONS

This application is a continuation application based on U.S. patent application Ser. No. 17/501,194, which was filed in the United States on Oct. 14, 2021, the entire contents of which are incorporated herein by reference.

### TECHNICAL FILED

The present invention relates to a method for calibrating an endoscope and an endoscope system.

### BACKGROUND

Conventionally, an endoscope system including an endoscope has been used for observation in a luminal organ such as a digestive tract and treatment in open surgery. The endoscope includes a curved portion capable of being driven by bending. Although the endoscope is supplied in a sufficiently calibrated state, it may be necessary to adjust the operation of the curved portion due to aging or the like. In that case, the user needs to calibrate the endoscope using a calibration jig, as described in PCT International Publication No. WO 2016/136353 and the like.

### SUMMARY

A method for calibrating an endoscope according to a first aspect of the present disclosure, wherein the endoscope including a curved portion that can be driven to bend and a flexible portion through which a bending wire that bends the curved portion is inserted, includes: arranging the curved portion and the flexible portion of the endoscope so that they have a predetermined shape; bending the curved portion; measuring first shape information, which is information about a shape of the curved portion; and updating a control parameter for the endoscope to drive the curved portion based on the measured first shape information.

The endoscope system according to a second aspect of the present disclosure includes: an endoscope including a curved portion that can be driven to bend, and a flexible portion through which a bending wire that bends the curved portion is inserted; and a control device including a processor that controls driving of the curved portion, wherein the processor is configured to perform: bending the curved portion; measuring first shape information, which is information about a shape of the curved portion; and updating a control parameter for the endoscope to drive the curved portion based on the measured first shape information.

### BRIEF DESCRIPTION OF THE DRAWINGS

FIG. 1 is an overall view of an electric endoscope system according to a first embodiment.

FIG. 2 is a diagram showing an endoscope and an operation device of the electric endoscope system used by the operator.

FIG. 3 is a diagram showing an insertion portion of the endoscope.

FIG. 4 is a cross-sectional view of a part of a curved portion of the endoscope.

FIG. 5 is an enlarged view of a knot ring of the curved portion in region E shown in FIG. 4.

FIG. 6 is a cross-sectional view of the curved portion along the C1-C1 line of FIG. 4 and FIG. 5.

FIG. 7 is a functional block diagram of a driving device of the electric endoscope system.

FIG. 8 is a functional block diagram of a video control device of the electric endoscope system.

FIG. 9 is a functional block diagram of a main controller of the electric endoscope system.

FIG. 10 is a control flowchart of a main controller of a control device in the electric endoscope system.

FIG. 11 is a diagram showing the endoscope with its distal end suspended.

FIG. 12 is a control flowchart of the main controller of the control device in the electric endoscope system according to a second embodiment.

FIG. 13 is a diagram showing a sheet-shaped marker as an example of a marker.

FIG. 14 is a diagram showing a box-shaped marker as an example of a marker.

FIG. 15 is an overall view of an electric endoscope system according to a third embodiment.

FIG. 16 is a diagram showing a reference model used by a drive controller of the electric endoscope system.

FIG. 17 is a diagram showing a pair of bending wires that pass through the bending insertion portion.

FIG. 18 is a diagram showing a pair of bending wires that pass through the bending insertion portion.

FIG. 19 is a diagram showing a pair of bending wires that pass through the bending insertion portion.

FIG. 20 is a diagram showing a pair of bending wires that pass through the bending insertion portion.

FIG. 21 is a diagram showing a pair of bending wires that pass through the bending insertion portion.

FIG. 22 is a control flowchart of a main controller of a control device of the electric endoscope system.

FIG. 23 is a graph showing a relationship between a measured tension and displacement of an upward bending wire.

FIG. 24 is a diagram showing another aspect of step S210 (arrangement step).

FIG. 25 is a diagram showing another aspect of step S210 (arrangement step).

FIG. 26 is an overall view of an electric endoscope system according to a fourth embodiment.

FIG. 27 is a diagram showing a packing box of the electric endoscope system.

FIG. 28 is a diagram showing a modification of the packing box.

FIG. 29 is a diagram showing another modification of the packing box.

### DETAILED DESCRIPTION

### First Embodiment

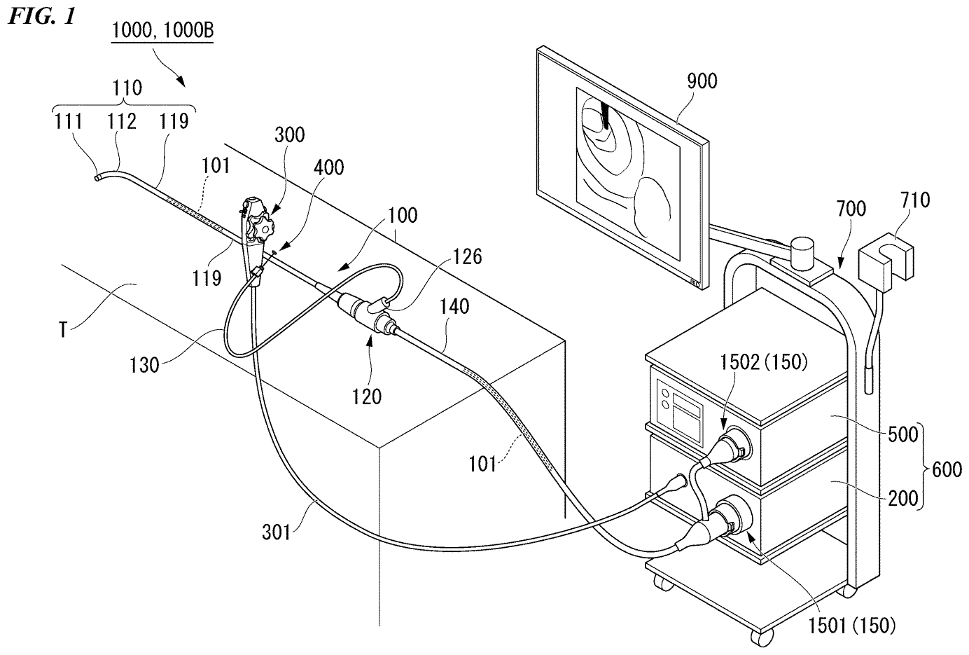

An electric endoscope system 1000 according to a first embodiment of the present disclosure will be described with reference to FIG. 1 to FIG. 17. FIG. 1 is an overall view of the electric endoscope system 1000 according to the present embodiment. The electric endoscope system 1000 is an example of a manipulator system.

### [Electric Endoscope System 1000]

The electric endoscope system 1000 is a medical system used to observe and treat the inside of a patient P as shown in FIG. 1. Further, the electric endoscope system 1000 is a medical system that maintains an endoscope 100. The electric endoscope system 1000 includes the endoscope 100, a driving device 200, an operation device 300, a treatment tool 400, a video control device 500, a display device 900, and an accommodation rack 700.

The endoscope 100 is a device that is inserted into the lumen of the patient P to observe and treat the affected area. The endoscope 100 is detachable from the driving device 200. An internal path 101 is formed inside the endoscope 100. In the following description, in the endoscope 100, the side inserted into the lumen of the patient P is referred to as the “distal end side (A1)”, and the side mounted on the driving device 200 is referred to as the “proximal end side (A2)”.

The driving device 200 is detachably connected to the endoscope 100 and the operation device 300. The driving device 200 drives the built-in motor to electrically drive the endoscope 100 based on the operation input to the operation device 300. Further, the driving device 200 drives a built-in pump or the like based on the operation input to the operation device 300 to cause the endoscope 100 to perform air supply suction.

The operation device 300 is detachably connected to the driving device 200 via an operation cable 301. The operation device 300 may be capable of communicating with the driving device 200 by wireless communication instead of wired communication. An operator S can electrically drive the endoscope 100 by operating the operation device 300.

The treatment tool 400 is a device for treating the affected portion by inserting the internal path 101 of the endoscope 100 into the lumen of the patient P. In FIG. 1, the treatment tool 400 is inserted into the internal path 101 of the endoscope 100 via an extension channel tube 130. The treatment tool 400 may be inserted directly from a forceps opening 126 into the internal path 101 of the endoscope 100 without passing through the extension channel tube 130.

The video control device 500 is detachably connected to the endoscope 100, and acquires a captured image from the endoscope 100. The video control device 500 causes the display device 900 to display the captured image acquired from the endoscope 100 and a GUI image or CG image to provide information to the operator.

The driving device 200 and the video control device 500 constitute a control device 600 that controls the electric endoscope system 1000. The control device 600 may further include a peripheral device such as a video printer. The driving device 200 and the video control device 500 may he an integrated device.

The display device 900 is a device capable of displaying an image such as an LCD. The display device 900 is connected to the video control device 500 via a display cable 901.

The driving device 200, the video control device 500, and the display device 900 are accommodated in the accommodation rack 700, The accommodation rack 700 is equipped with tires and is easy to move. The accommodation rack 700 is provided with a hanger (trolley) 710 that can he installed by suspending the endoscope 100.

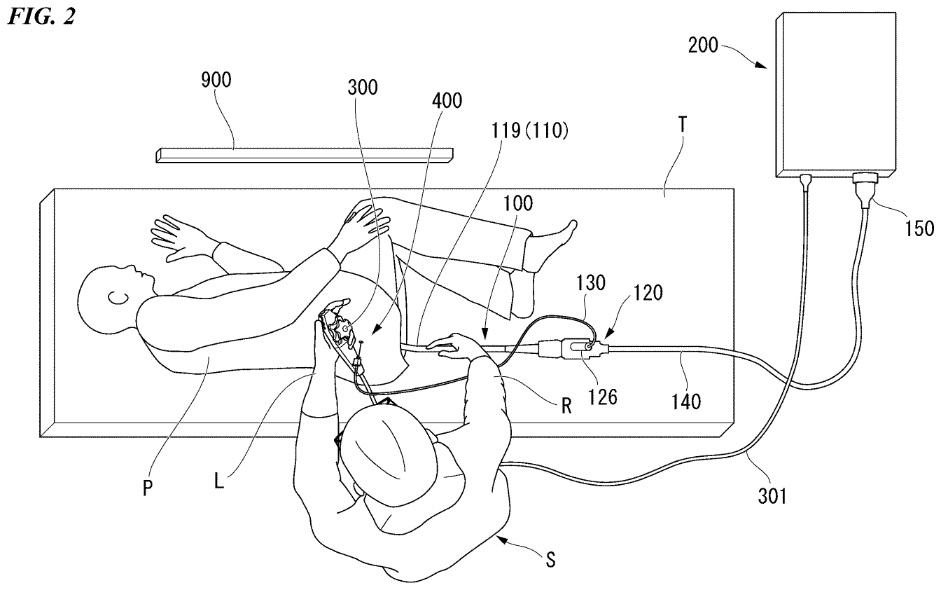

FIG. 2 is a diagram showing the endoscope 100 and the operation device 300 used by the operator S.

For example, the operator S operates the endoscope 100 inserted into the lumen from the anus of the patient P with the right hand R while observing the captured image displayed on the display device 900, and operates the operation device 300 with the left hand L. Since the endoscope 100 and the operation device 300 are separated, the operator S can operate the endoscope 100 and the operation device 300 independently without either affecting the other.

### [Endoscope 1001]

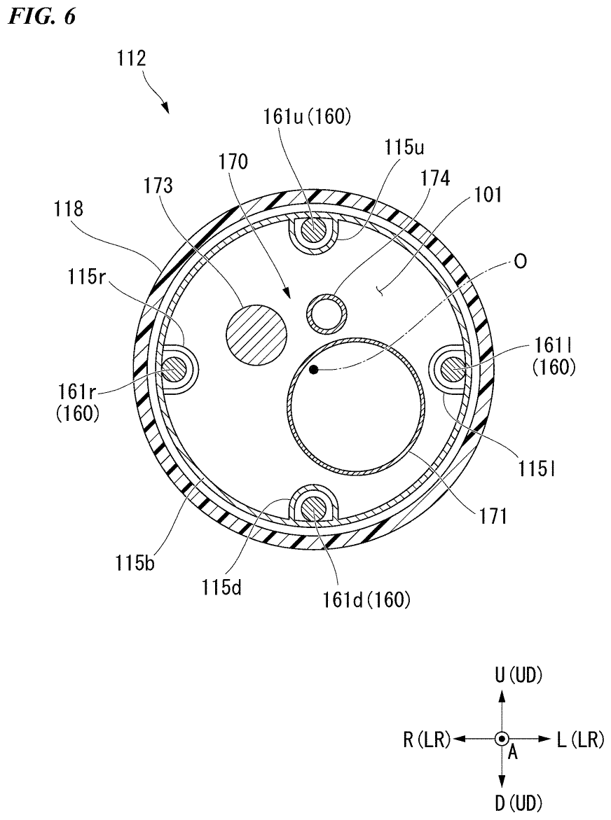

As shown in FIG. 1, the endoscope 100 includes an insertion portion 110, a connecting portion 120, an extracorporeal flexible portion 140, an attachment/detachment portion 150, a curved wire 160 (see FIG. 6), and an internal object 170 (see FIG. 6). The insertion portion 110, the connecting portion 120, the extracorporeal flexible portion 140, and the attachment/detachment portion 150 are connected in order from the distal end side. The connecting portion 120 can connect the extension channel tube 130.

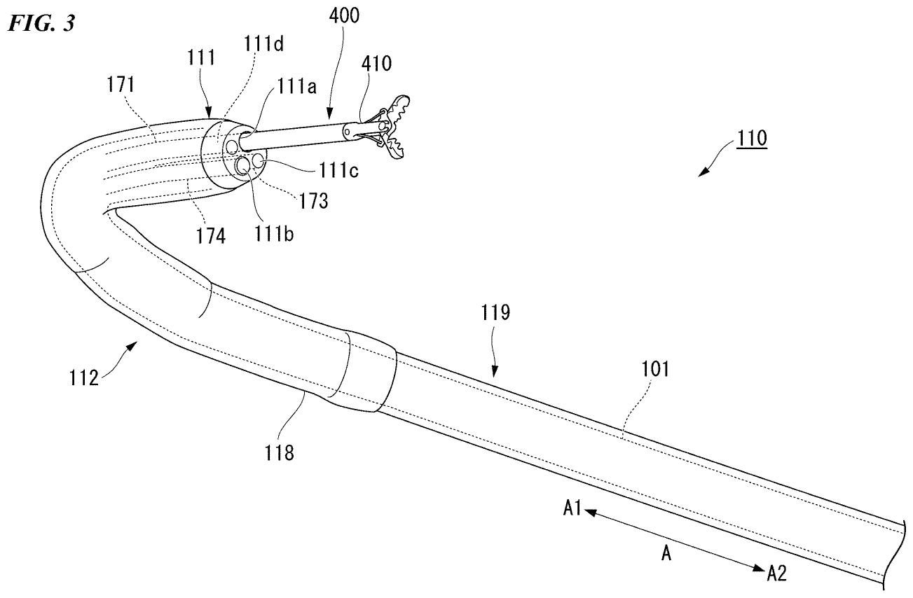

FIG. 3 is a diagram showing the insertion portion 110 of the endoscope 100.

Inside the endoscope 100, the internal path 101 is formed that extends from the distal end of the insertion portion 110 to the proximal end of the attachment/detachment portion 150 along the longitudinal direction A of the endoscope 100. The curved wire 160 and the internal object 170 are inserted into the internal path 101.

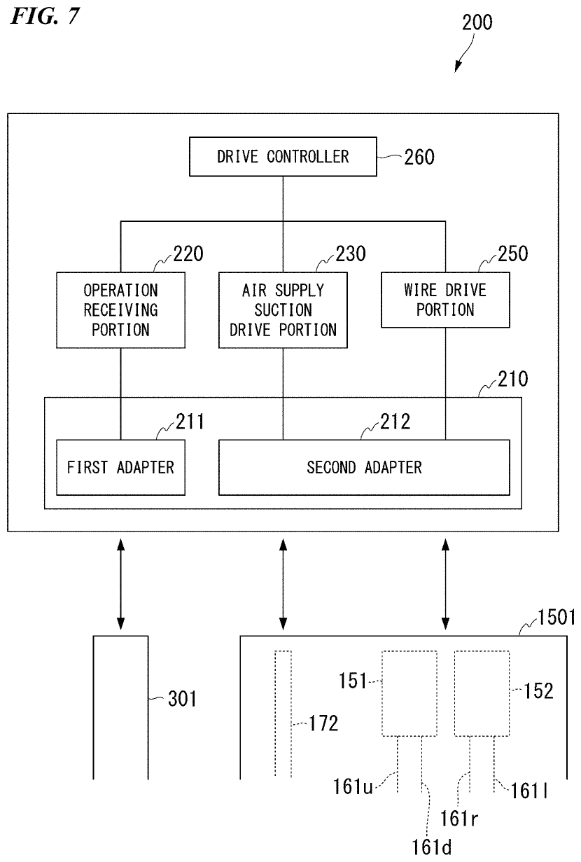

The internal object 170 includes a channel tube 171, an air supply suction tube 172 (see FIG. 7), an imaging cable 173, and a light guide 174.

### [Insertion Portion 110]

The insertion portion 110 is an elongated long member that can be inserted into the lumen. The insertion portion 110 has a distal end portion 111, a curved portion 112, and an internal flexible portion 119. The distal end portion 111, the curved portion 112, and the internal flexible portion 119 are connected in order from the distal end side.

The distal end portion 111 is formed of a metal or the like into a substantially cylindrical shape. As shown in FIG. 3, the distal end portion 111 has an opening portion 111a, an illumination portion 111b, and an imaging portion 111c. The opening portion 111a is an opening that communicates with the channel tube 171. As shown in FIG. 3, a treatment portion 410 such as a gripping forceps provided at the distal end of the treatment tool 400 through which the channel tube 171 is inserted protrudes from the opening portion 111a.

The illumination portion 111b is connected to the light guide 174 that guides the illumination light, and emits illumination light that illuminates an imaging target. The imaging portion 111c includes an imaging element such as CMOS, and images the imaging target. An imaging signal is transmitted to the video control device 500 via the imaging cable 173.

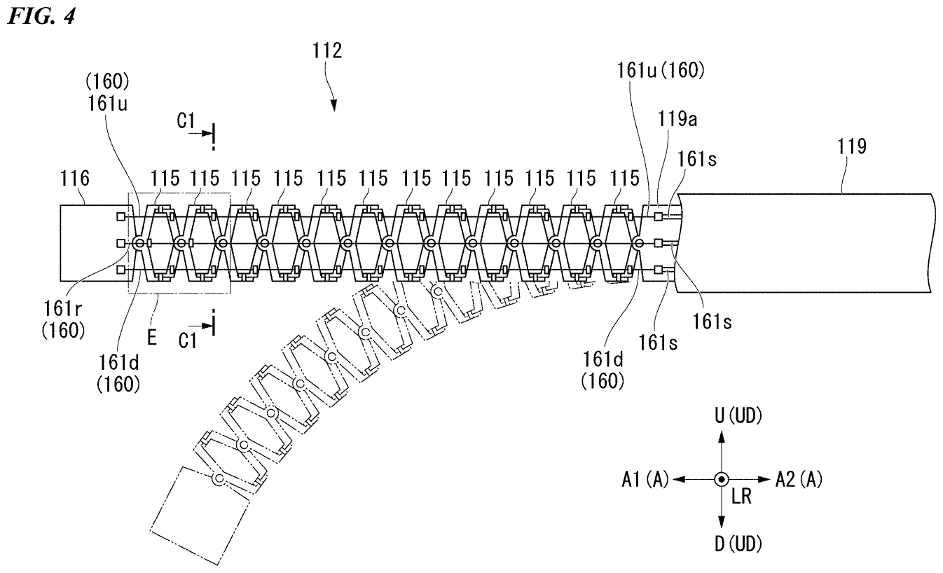

FIG. 4 is a view showing a part of the curved portion 112 as a cross-sectional view.

The curved portion 112 includes a plurality of knot rings (also referred to as curved pieces) 115, a distal end portion 116 connected to the distal ends of the plurality of knot rings 115, and an outer sheath 118 (see FIG. 3). The distal end portion 116, the plurality of knot rings 115, and a distal end portion 119a of the internal flexible portion 119 are connected in the longitudinal direction A inside the outer sheath 118. The shape and number of the knot rings 115 included in the curved portion 112 are not limited to the shape and number of the knot rings 115 shown in FIG. 4.

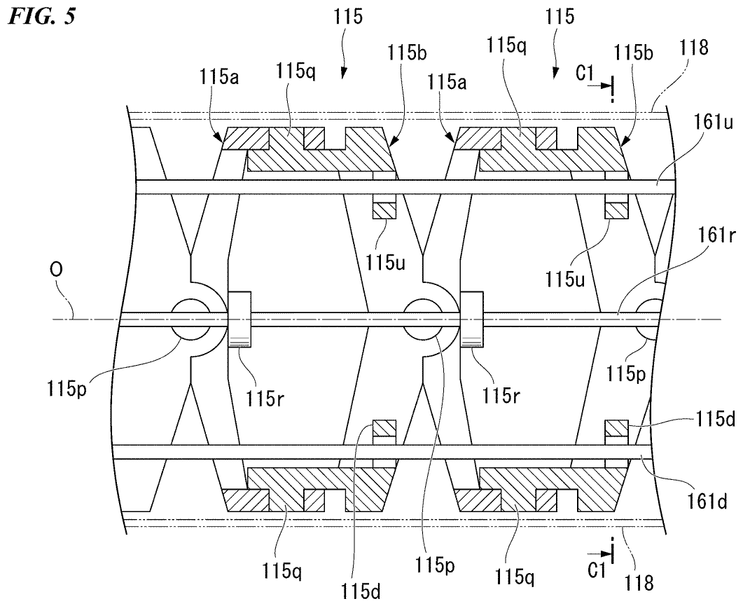

FIG. 5 is an enlarged view of the knot ring 115 in the region E shown in FIG. 4.

The knot ring 115 is a short cylindrical member made of metal. The plurality of knot rings 115 are connected so that the internal spaces of the adjacent knot rings 115 are continuous spaces.

Adjacent knot rings 115 are rotatably connected by a first rotation pin 115p in a vertical direction (also referred to as “UD direction”) perpendicular to the longitudinal direction A.

The knot ring 115 has a first knot ring 115a on the distal end side and a second knot ring 115b on the proximal end side. The first knot ring 115a and the second knot ring 115b are rotatably connected by a second rotation pin 115q in the left-right direction (also referred to as “LR direction”) perpendicular to the longitudinal direction A and the UD direction.

The first rotation pin 115p of the knot ring (curved piece) 115 is rotatable about a rotation axis extending in the LR direction. The second rotation pin 115q of the knot ring (curved piece) 115 is rotatable about a rotation axis extending in the UD direction.

The first knot ring 115a and the second knot ring 115b are alternately connected by the first rotation pin 115p and the second rotation pin 115q, and the curved portion 112 is bendable in a desired direction.

FIG. 6 is a cross-sectional view of the curved portion 112 along the C1-C1 line of FIG. 4 and FIG. 5.

An upper wire guide 115u and a lower wire guide 115d are formed on the inner peripheral surface of the second knot ring 115b. The upper wire guide 115u and the lower wire guide 115d are arranged on both sides in the UD direction with the central axis O in the longitudinal direction A interposed therebetween. A left wire guide 115l and a right wire guide 115r are formed on the inner peripheral surface of the first knot ring 115a. The left wire guide 115l and the right wire guide 115r are arranged on both sides in the LR direction with the central axis O in the longitudinal direction A interposed therebetween.

Through-holes through which the curved wire 160 is inserted are formed in the upper wire guide 115u, the lower wire guide 115d, the left wire guide 115l, and the right wire guide 115r along the longitudinal direction A.

The curved wire 160 is a wire that bends the curved portion 112. The curved wire 160 extends to the attachment/detachment portion 150 through the internal path 101. As shown in FIG. 4 and FIG. 6, the curved wire 160 has an upper curved wire 161u, a lower curved wire 161d, a left curved wire 161l, a right curved wire 161r, and four wire sheaths 161s.

As shown in FIG. 4, separate wire sheaths 161s are inserted into the upper curved wire 161u, the lower curved wire 161d, the left curved wire 161l, and the right curved wire 161r. The distal end of the wire sheath 161s is attached to the knot ring 115 at the proximal end of the curved portion 112. The wire sheath 161s extends to the attachment/detachment portion 150.

The upper curved wire 161u and the lower curved wire 161d are wires that bend the curved portion 112 in the UD direction. The upper curved wire 161u is inserted with the upper wire guide 115u. The lower curved wire 161d is inserted with the lower wire guide 115d.

As shown in FIG. 4, the distal ends of the upper curved wire 161u and the lower curved wire 161d are fixed to the distal end 116 of the distal end of the curved portion 112. The distal ends of the upper curved wire 161u and the lower curved wire 161d fixed to the distal end portion 116 are arranged on both sides in the UD direction with the central axis O in the longitudinal direction A interposed therebetween.

The left curved wire 161l and the right curved wire 161r are wires that bend the curved portion 112 in the LR direction. The left curved wire 161l is inserted with the left wire guide 115l. The right curved wire 161r is inserted with the right wire guide 115r.

The distal ends of the left curved wire 161l and the right curved wire 161r are fixed to the distal end 116 of the curved portion 112 as shown in FIG. 4. The distal ends of the left-curved wire 161l and the right-curved wire 161r fixed to the distal end portion 116 are arranged on both sides in the LR direction with the central axis O in the longitudinal direction A interposed therebetween.

The curved portion 112 can be bent in a desired direction by pulling or relaxing the curved wire 160 (upper curved wire 161u, lower curved wire 161d, left curved wire 161l, right curved wire 161r).

As shown in FIG. 6, the curved wire 160, the channel tube 171, the imaging cable 173, and the light guide 174 are inserted into the internal path 101 formed inside the curved portion 112.

The internal flexible portion 119 is a long and flexible tubular member. The curved wire 160, the channel tube 171, the imaging cable 173, and the light guide 174 are inserted into an internal path 101 formed in the internal flexible portion 119. At the distal end of the internal flexible portion 119, a distal end portion 119a formed in a substantially cylindrical shape by a metal or the like is provided.

### [Connecting Portion 120]

As shown in FIG. 1, the connecting portion 120 is a member that connects the internal flexible portion 119 and the extracorporeal flexible portion 140 of the insertion portion 110. The connecting portion 120 includes the forceps opening 126, which is an insertion port for inserting the treatment tool 400 into the internal path 101.

### [Extracorporeal Flexible Portion 140]

The extracorporeal flexible portion 140 is a long tubular member. The curved wire 160, the imaging cable 173, the light guide 174, and the air supply suction tube 172 (see FIG. 7) are inserted into the internal path 101 formed inside the extracorporeal flexible portion 140.

### [Attachment/Detachment Portion 150]

As shown in FIG. 1, the attachment/detachment portion 150 includes a first attachment/detachment portion 1501 attached to the driving device 200 and a second attachment/detachment portion 1502 attached to the video control device 500. The first attachment/detachment portion 1501 and the second attachment/detachment portion 1502 may be an integral attachment/detachment portion.

The internal path 101 formed inside the extracorporeal flexible portion 140 branches into the first attachment/detachment portion 1501 and the second attachment/detachment portion 1502. The curved wire 160 and the air supply suction tube 172 are inserted into the first attachment/detachment portion 1501. The imaging cable 173 and the light guide 174 are inserted into the second attachment/detachment portion 1502.

The first attachment/detachment portion 1501 has a tension sensor (not shown) that detects the tension of the curved wire 160 (upper curved wire 161u, lower curved wire 161d, left curved wire 161l, right curved wire 161r). The detection result of the tension sensor is acquired by a drive controller 260 of the driving device 200.

### [Driving Device 200]

FIG. 7 is a functional block diagram of the driving device 200.

The driving device 200 includes an adapter 210, an operation-receiving portion 220, an air supply suction drive portion 230, a wire drive portion 250, and the drive controller 260.

The adapter 210 has a first adapter 211 and a second adapter 212. The first adapter 211 is an adapter to which an operation cable 301 is detachably connected. The second adapter 212 is an adapter to which the first attachment/detachment portion 1501 of the endoscope 100 is detachably connected.

The operation-receiving portion 220 receives an operation input from the operation device 300 via the operation cable 301. In the case where the operation device 300 and the driving device 200 communicate by wireless communication instead of wired communication, the operation-receiving portion 220 includes a known wireless-receiving module.

The air supply suction drive portion 230 is connected to the air supply suction tube 172 inserted into the internal path 101 of the endoscope 100. The air supply suction drive portion 230 includes a pump and the like, and supplies air to the air supply suction tube 172. Further, the air supply suction drive portion 230 sucks air from the air supply suction tube 172.

The wire drive portion 250 has a drive portion and an encoder (not shown). The drive portion pulls or relaxes the curved wire 160 (upper curved wire 161u, lower curved wire 161d, left curved wire 161l, right curved wire 161r) by a pulley or the like. The encoder detects a traction amount of the curved wire 160. The detection result of the encoder is acquired by the drive controller 260 of the driving device 200.

The drive controller 260 controls the entire driving device 200. The drive controller 260 acquires the operation input received by the operation-receiving portion 220. The drive controller 260 controls the air supply suction drive portion 230 and the wire drive portion 250 based on the acquired operation input. Specifically, the drive controller 260 calculates a control amount (wire traction amount, wire tension, or the like) in which the wire drive portion 250 drives the curved portion 112 or the like, from the operation input to the curved portion 112 or the like received by the operation-receiving portion 220, based on a changeable control parameter and a conversion formula having the control parameter as a coefficient. The control parameter is not limited to the coefficient of the conversion formula, and may be a parameter that replaces a part or all of the conversion formula.

The drive controller 260 is a computer capable of executing a program including a processor, a memory, a storage capable of storing programs and data, and an input/output controller. The function of the drive controller 260 is realized by the processor executing the program. At least some functions of the drive controller 260 may be realized by a dedicated logic circuit.

Since the drive controller 260 controls a plurality of motors for driving a plurality of curved wires 160 with high accuracy, it is desirable that the drive controller 260 have high calculation performance.

The drive controller 260 may further have a configuration other than the processor, the memory, the storage, and the input/output controller. For example, the drive controller 260 may further have an image calculation portion that performs some or all of the image processing and image recognition processing. By further having the image calculation portion, the drive controller 260 can execute specific image processing and image recognition processing at high speed. The image calculation portion may be mounted on a separate hardware device connected by a communication line.

### [Operation Device 300]

The operation device 300 is a device for inputting an operation for driving the endoscope 100. The input operation input is transmitted to the driving device 200 via the operation cable 301.

### [Video Control Device 500]

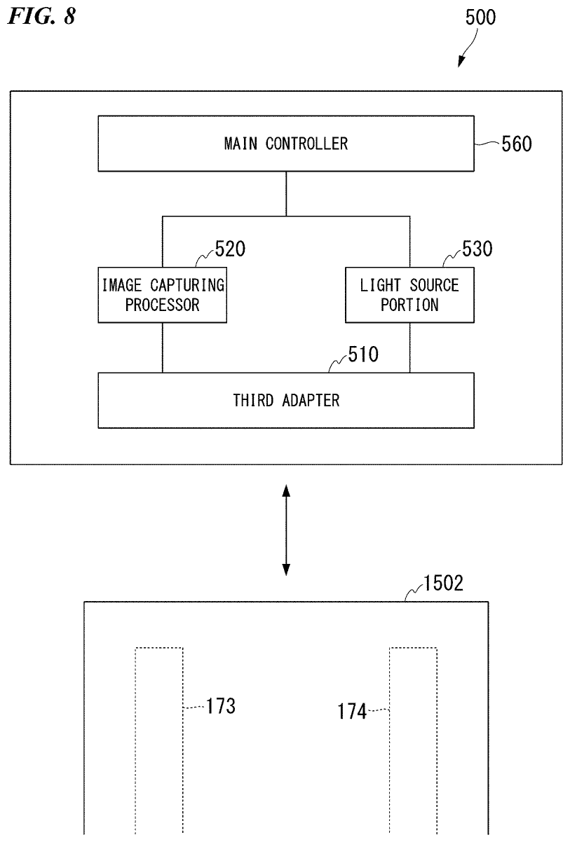

FIG. 8 is a functional block diagram of the video control device 500.

The video control device 500 controls the electric endoscope system 1000. The video control device 500 includes a third adapter 510 an imaging processor 520, a light source portion 530, and a main controller 560.

The third adapter 510 is an adapter to which the second attachment/detachment portion 1502 of the endoscope 100 is detachably connected.

The imaging processor 520 converts the imaging signal acquired from the imaging portion 111c of the distal end portion 111 into a captured image via the imaging cable 173.

The light source portion 530 generates illumination light to be applied to the imaging target. The illumination light generated by the light source portion 530 is guided to the illumination portion 111b of the distal end portion 111 via the light guide 174.

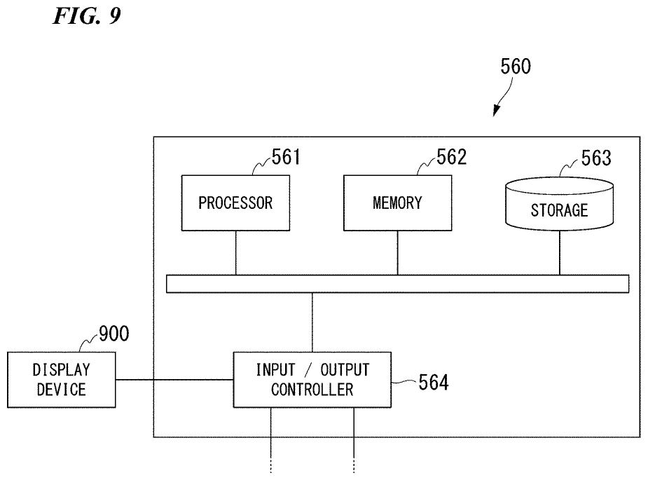

FIG. 9 is a functional block diagram of the main controller 560.

The main controller 560 is a computer capable of executing a program equipped with a processor 561 and a memory 562 or the like. The function of the main controller 560 is realized by the processor 561 executing the program. At least some functions of the main controller 560 may be realized by a dedicated logic circuit.

The main controller 560 has a processor 561, a memory 562 that can read the program, a storage 563, and an input/output controller 564.

The storage 563 is a non-volatile recording medium that stores the above-mentioned program and necessary data. The storage 563 is composed of, for example, a ROM, a hard disk, or the like. The program stored in the storage 563 is read into the memory 562 and executed by the processor 561.

The input/output controller 564 is connected to the imaging processor 520, the light source portion 530, the driving device 200, a measurement device 800, the display device 900, an input device (not shown), an external camera (not shown), and a network device (not shown). The input output controller 564 transmits/receives data to/from the connected device and transmits/receives control signals based on the control of the processor 561.

The main controller 560 can perform image processing on the captured image acquired by the imaging processor 520. The main controller 560 can generate a GUI image or a CG image to provide information to the operator S. The main controller 560 can display the captured image, GUI image, and CG image on the display device 900.

The main controller 560 is not limited to an integrated hardware device. For example, the main controller 560 may be configured by partially separating the hardware devices as separate hardware devices and then connecting the separated hardware devices via a communication line. For example, the main controller 560 may be a cloud system that connects the separated storage 563 with a communication line.

The main controller 560 may further have a configuration other than the processor 561, the memory 562, the storage 563, and the input/output controller 564 shown in FIG. 9. For example, the main controller 560 may further have the image calculation portion that performs some or all of the image processing and the image recognition processing performed by the processor 561. By further having the image calculation portion, the main controller 560 can execute specific image processing and image recognition processing at high speed. The image calculation portion may be mounted on a separate hardware device connected by a communication line.

### [Operation Of Electric Endoscope System 1000]

Next, the operation of the electric endoscope system 1000 of this embodiment will be described. Specifically, the operation of the user calibrating the endoscope 100 before use in the operating room or the treatment room will be described.

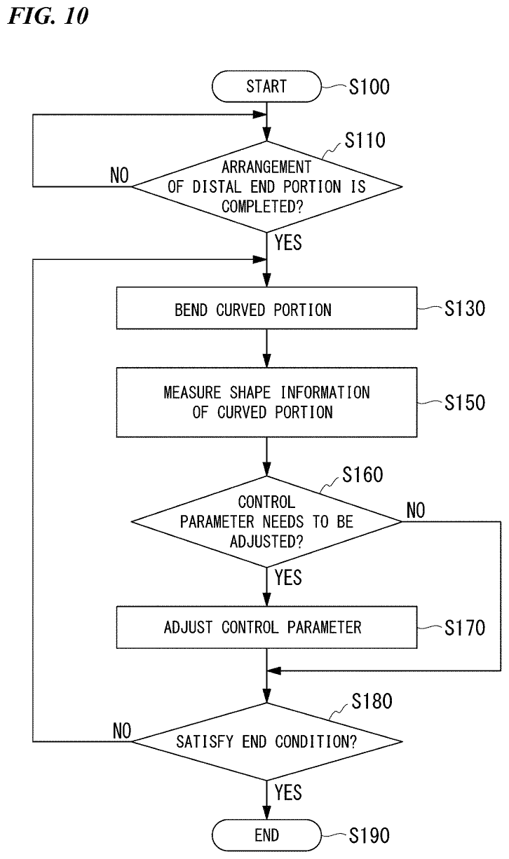

Hereinafter, a description will be given according to the control flowchart of the main controller 560 of the control device 600 shown in FIG. 10. When the control device 600 is activated, the main controller 560 starts control after performing initialization (step S100). Next, the main controller 560 (mainly the processor 561) performs step S110.

### <Step S110: Arrangement Step>

In step S110, the main controller 560 displays the GUI image on the display device 900 instructing the user to hang the endoscope 100 on the hanger 710 and suspend a distal end portion 180 of the endoscope 100 including the curved portion 112 from the hanger 710.

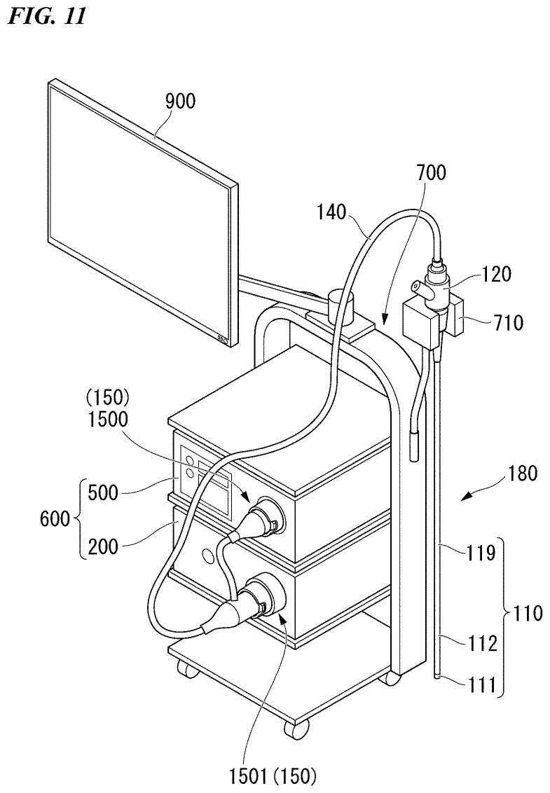

FIG. 11 is a diagram showing an endoscope 100 or the like in which the distal end portion 180 is suspended.

The user hangs the distal end portion 180 from the hanger 710 according to the instructions displayed on the GUI image. Specifically, the user suspends and arranges the distal end portion 180 so that the distal end portion 180 does not come into contact with the floor or the like and the distal end portion 180 is in a straight line state along the vertical direction. In the following description, the state of the suspended distal end portion 180 is also referred to as an “initial state” or a “natural hanging state”. It should be noted that the linear state includes a state in which the insertion portion 110 naturally assumes a shape when the insertion portion 110 is suspended and is not subjected to external force, and is not necessarily limited to the completely straight state of the insertion portion 110.

The distal end portion 180 is a portion of the distal end side A1 of the endoscope 100 including at least the curved portion 112. The distal end portion 180 may include part or all of the internal flexible portion 119. Further, a part or all of the extracorporeal flexible portion 140 may be included.

In the example shown in FIG. 11, the connecting portion 120 is hung on the hanger 710, and the insertion portion 110 is hung as the distal end portion 180. The internal flexible portion 119 may be hung on the hanger 710, and a part of the internal flexible portion 119, the curved portion 112, and the distal end portion 111 may be suspended as the distal end portion 180. The extracorporeal flexible portion 140 may be hung on the hanger 710, and a part of the extracorporeal flexible portion 140, the internal flexible portion 119, the curved portion 112, and the distal end portion 111 may he suspended as the distal end portion 180.

The main controller 560 detects whether the distal end portion 180 of the endoscope 100 is in the initial state. The main controller 560 may detect whether the distal end portion 180 is in the initial state by causing the user to input information indicating the completion of the arrangement step from an input device (not shown). Further, the main controller 560 may detect whether the distal end portion 180 is in the initial state based on the captured image captured by the imaging portion 111c of the endoscope 100, the sensor mounted on the endoscope 100, or the like. Further, the main controller 560 may detect whether the distal end portion 180 is in the initial state based on an image taken by an external camera (not shown) connected to the video control device 500. Next, the main controller 560 performs step S130.

### <Step S130: Bending Step >

In step S130, the main controller 560 controls the drive controller 260 so that the control amount (wire traction amount, wire tension, or the like) of the curved portion 112 is the adjustment value V, and bends the curved portion 112 in at least one direction of the UD direction and the LR direction. Next, the main controller 560 performs step S150.

In step S130, the main controller 560 may estimate the bending direction in which the curved portion 112 is frequently used and is greatly affected by aging deterioration from the usage history of the endoscope 100, so as to bend the curved portion 112 in the estimated bending direction. As a result, the main controller 560 can efficiently calibrate the endoscope 100.

### <Step S150: Measurement Step>

The main controller 560 measures the shape information of the curved portion 112 in step S150, Specifically, the main controller 560 measures the shape information of the curved portion 112 based on the captured image captured by the imaging portion 111c of the endoscope 100, the sensor mounted on the endoscope 100, and the like. Further, the main controller 560 may evaluate the shape information of the curved portion 112 based on an image taken by an external camera (not shown) connected to the video control device 500. Next, the main controller 560 performs step S160.

### <Step S160: Adjustment Step>

The main controller 560 compares the shape information of the curved portion 112 that is ideally curved when the control amount (wire traction amount, wire tension, or the like) is set to the adjustment value V in step S160, and the shape information measured in step S150. Here, the main controller 560 holds in advance the shape information of the curved portion 112 that is ideally curved when the control amount is set to a predetermined value.

When the compared shape information is different, the main controller 560 determines that the control parameter for driving the curved portion 112 by the drive controller 260 of the endoscope 100 needs to be updated, and then performs step S170. In the case where there is almost no difference in the compared shape information, the main controller 560 determines that it is not necessary to update the control parameters, and then performs step S180.

### <Step S170: Adjustment Step>

The main controller 560 updates a control parameter used when calculating the control amount (wire traction amount, wire tension, or the like) required for the drive controller 260 of the endoscope 100 to form the curved portion 112 into a predetermined curved shape based on the shape information measured in the measurement step in step S170. The control parameters may be possessed by the drive controller 260 or the main controller 560.

The main controller 560 selects an optimum control parameter from a plurality of control parameters prepared in advance based on the above shape information. For example, the main controller 560 may select the optimum control parameter by using a learned model (machine learning model) in which the relationship between the shape information and the control parameter is learned in advance by machine learning. Further, the main controller 560 may select the optimum control parameter by referring to a database in which the relationship between the shape information and the control parameter is recorded in advance. Next, the main controller 560 performs step S180.

### <Step S180: End Determination Step>

In step S180, the main controller 560 determines the end of calibration of the endoscope 100. For example, in the case where a calibration step (bending step, measurement step, and adjustment step) is performed in each of the UD direction and the LR direction, the main controller 560 determines that the calibration of the endoscope 100 has been completed, and performs step S190 to end the control. In the case where it is determined that the calibration of the endoscope 100 has not been completed, the main controller 560 performs step S130 again.

According to the electric endoscope system 1000 of the present embodiment, the endoscope 100 can be easily calibrated without using a special calibration jig for fixing the curved portion 112, in a state where the endoscope 100 is normally stored, that is, in a state where the endoscope 100 is hung on the hanger 710. Therefore, even when the operating room or treatment room in which the electric endoscope system 1000 is installed is small, the control device 600 (mainly the main controller 560) can easily calibrate the endoscope 100.

The distal end portion 180 of the endoscope 100 is suspended so as to be in a straight line along the vertical direction in the initial state. Therefore, the shape of the distal end portion 180 of the endoscope 100 in the initial state is uniquely determined without using a special calibration jig. Therefore, since the control device 600 (mainly the main controller 560) can perform the calibration step based on the uniquely determined initial shape, the endoscope 100 can be accurately calibrated under the same conditions regardless of the environment in which it is used or the user Who uses it.

Although the first embodiment of the present disclosure has been described in detail with reference to the drawings, the specific configuration is not limited to this embodiment, and includes design changes and the like within a range that does not deviate from the gist of the present disclosure. In addition, the components shown in the above-described embodiment and modifications can be appropriately combined and configured.

### Second Embodiment

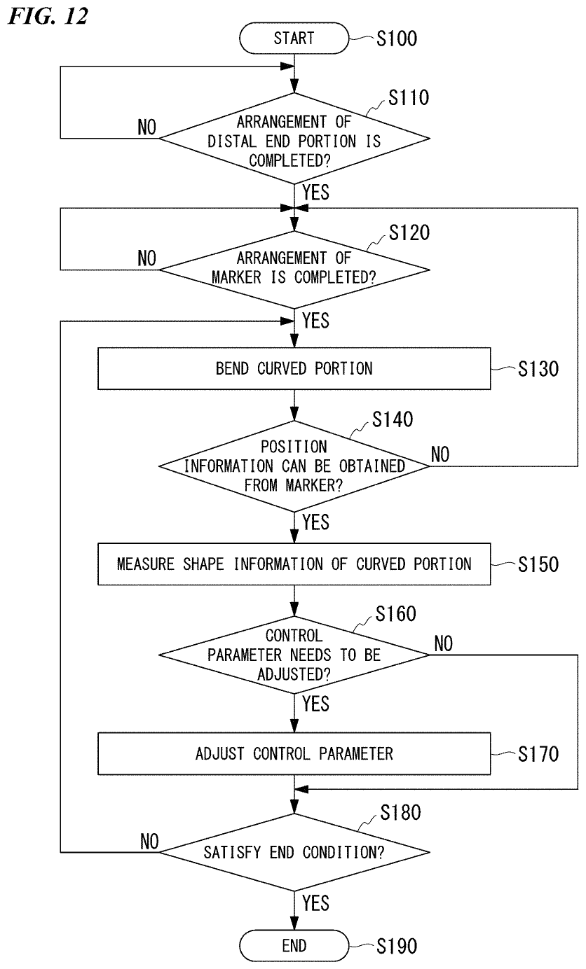

An electric endoscope system 1000B according to a second embodiment of the present disclosure will be described with reference to FIG. 12 to FIG. 14. In the following description, the same reference numerals will be given to the configurations common to those already described, and duplicate description will be omitted.

### [Electric Endoscope System 1000B]

As shown in FIG. 1, the electric endoscope system 1000B has the same configuration as the electric endoscope system 1000 of the first embodiment. The electric endoscope system 1000B differs from the electric endoscope system 1000 of the first embodiment only in the operation of using a marker.

Hereinafter, a description will be given according to the control flowchart of the main controller 560 of the control device 600 shown in FIG. 12. When the control device 600 is activated, the main controller 560 starts control after performing initialization (step S100). Next, the main controller 560 (mainly the processor 561) performs step S110.

### <Step S110: Arrangement Step>

In step S110, the main controller 560 implements the arrangement step in the same manner as in the first embodiment. Next, the main controller 560 performs step S120.

<Step S120: Marker Arrangement Step>

In step S120, the main controller 560 displays the GUI image instructing the user to arrange a marker M on the display device 900. The marker M has a known marker pattern m capable of specifying relative position information. The marker pattern m is a pattern in which relative position information can be specified by observing from different places.

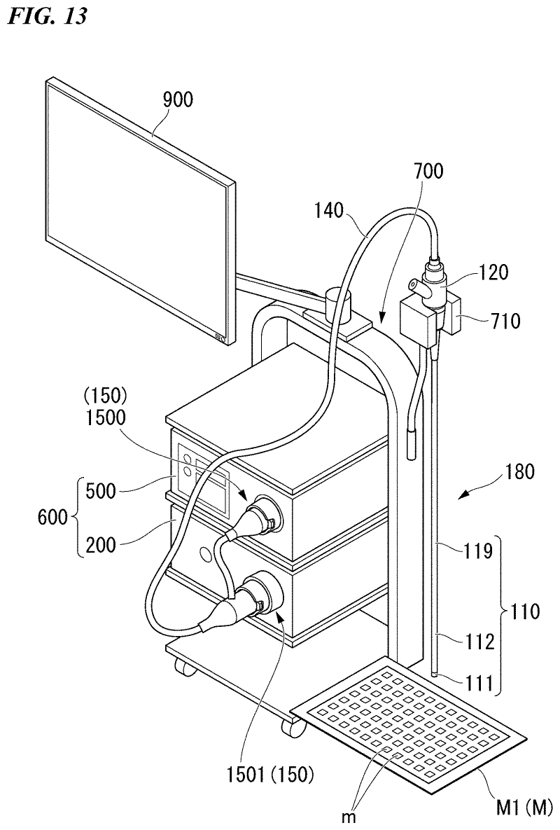

FIG. 13 is a diagram showing a sheet-shaped marker M1 as an example of the marker M.

The marker M1 is a sheet-shaped marker having a known marker pattern m. The user installs the marker M1 below the suspended distal end portion 180 in the vertical direction so as to form a straight line along the vertical direction. Since the field of view of the imaging portion 111c of the endoscope 100 is downward in the vertical direction, the imaging portion 111c can image the marker M1 and acquire relative position information from the marker M.

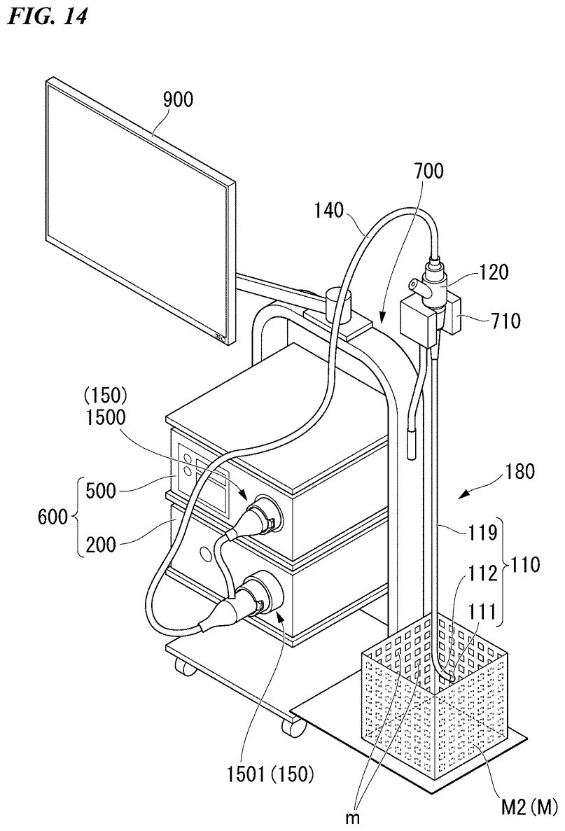

FIG. 14 is a diagram showing a box-shaped marker M2 as an example of the marker M.

The marker M installed by the user may be a box-shaped marker M2 having a plurality of surfaces having the marker pattern m. The marker M2 is formed in a box shape that opens upward in the vertical direction, and has a known marker pattern m on the inner surface. The user installs the marker M2 vertically below the suspended distal end portion 180 so that the distal end portion 111 of the endoscope 100 is arranged inside the box-shaped marker M2. Since the field of view of the imaging portion 111c of the endoscope 100 is downward in the vertical direction, the imaging portion 111c can image the marker M2 and acquire relative position information from the marker M.

The main controller 560 detects that the marker M has been arranged. The main controller 560 may detect that the marker M has been arranged by causing the user to input information indicating the completion of the marker arrangement step from an input device (not shown). Further, the main controller 560 may detect that the marker M is arranged based on the captured image or the like captured by the imaging portion 111c of the endoscope 100. Next, the main controller 560 performs step S130.

### <Step S130: Bending Step >

The main controller 560 performs the bending step in step S130 as in the first embodiment. Next, the main controller 560 performs step S140.

### <Step S140: Marker Position Information Acquisition Step>

In step S140, the main controller 560 images the marker M by the imaging portion 111c, and acquires the relative position information from the marker M based on the captured image. In the case where the imaging portion 111c cannot image the marker M or cannot acquire the relative position information from the marker M, there is a possibility that the position of the marker M is inadequate, so the main controller 560 displays the GUI image instructing the user to reposition the marker M on the display device 900, and performs step S120 again. The user repositions the marker M so that the marker pattern in is included in the field of view of the imaging portion 111c both before and after the curved portion 112 is curved. For example, the main controller 560 may estimate the direction in which the curved portion 112 is frequently used and is greatly affected by aging deterioration from the usage history of the endoscope 100, and display the GUI image on the display device 900 instructing the user to reposition the marker M so that the marker pattern m is included in the field f view of the imaging unit 111c before and after bending in the estimated. bending direction.

In the case where the curved portion 112 is greatly curved in the bending step. the marker M is preferably a box-shaped marker M2 rather than a sheet-shaped marker M1. This is because in the box-shaped marker M2, there is a high possibility that the marker pattern m is included in the field of view of the imaging portion 111c both before and after the curved portion 112 is curved.

If the main controller 560 can acquire the relative position information from the marker M in step S140, then the main controller 560 performs step S150.

### <Step S150: Measurement Step>

The main controller 560 measures the shape information of the curved portion 112 in step S150. Specifically, the shape information of the curved portion 112 is measured by using the relative position information from the acquired marker M. Next, the main controller 560 performs step S160 and subsequent steps as in the first embodiment.

According to the electric endoscope system 1000B of this embodiment, since the control device 600 (mainly the main controller 560) can perform the calibration step based on the uniquely determined initial shape, the endoscope 100 is accurately calibrated under the same conditions regardless of the environment in which it is used or the user who uses it. Further, the control device 600 (mainly the main controller 560) can evaluate the shape information of the curved portion 112 more accurately by using the marker M, and can calibrate the endoscope 100 more accurately.

The second embodiment of the present disclosure has been described in detail with reference to the drawings, but the specific configuration is not limited to this embodiment, and includes design changes and the like within a range that does not deviate from the gist of the present disclosure. In addition, the components shown the above-described embodiment and modifications can be appropriately combined and configured.

### Modification Example 1

In the above embodiment, the endoscope 100 of the electric endoscope system 1000 and the operation device 300 are separated. However, the modes of the endoscope 100 and the operation device 300 are not limited to this. The method for calibrating an endoscope according to the present disclosure may be applied to a conventional endoscope system in which an endoscope and an operation portion are integrated. In that case, for example, the operating portion of the conventional endoscope is hung on the hanger 710, and the insertion portion on the distal end side is hung as the distal end portion.

### Third Embodiment

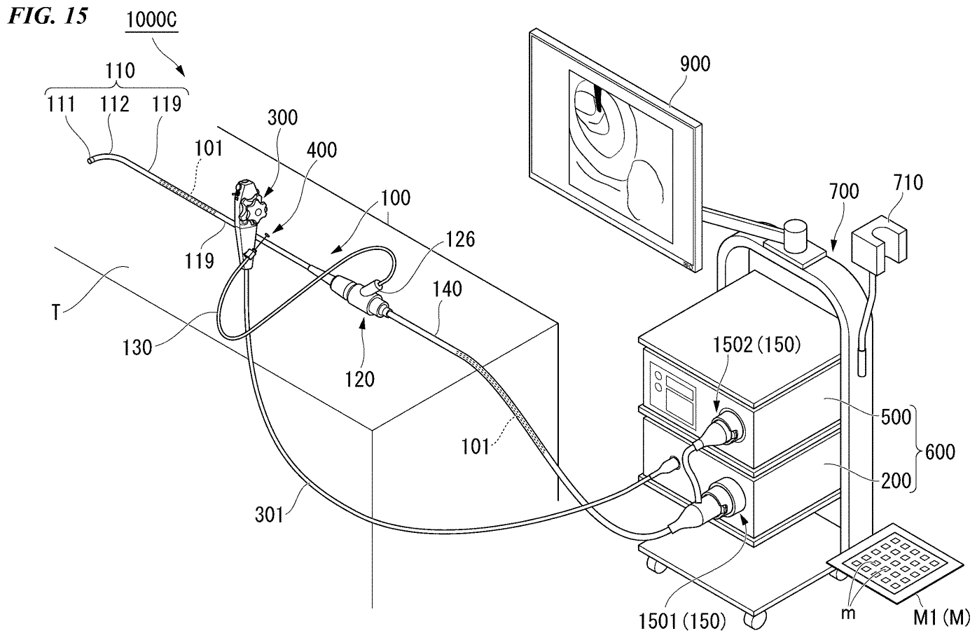

An electric endoscope system 1000C according to a third embodiment of the present disclosure will he described with reference to FIGS. 15 to 23. In the following description, the same reference numerals are given to the same configurations as those already described, and redundant descriptions will be omitted.

### [Electric Endoscope System 1000C]

FIG. 15 is an overall view of the electric endoscope system 1000C according to this embodiment.

The electric endoscope system 1000C includes an endoscope 100, a driving device 200, an operation device 300, a treatment tool 400, an image control device 500, a display device 900, and a storage rack 700.

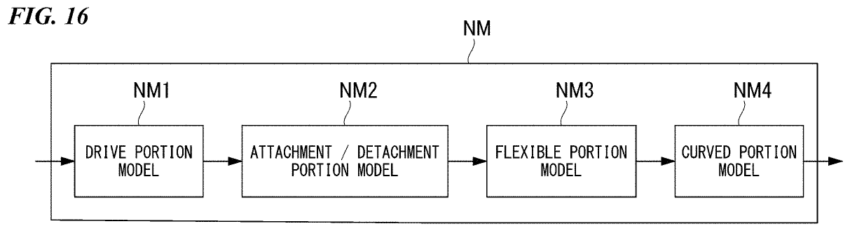

FIG. 16 is a diagram showing a reference model NM used by a drive controller 260.

The reference model NM is, for example, a model for estimating the bending motion of the endoscope 100. The reference model NM is used, for example, to calculate a motor current command value for the wire drive portion 250 required to bend the curved portion 112 by a desired amount. The reference model NM includes a drive portion model NM1 that models the wire drive portion 250, an attachment detachment portion model NM2 that models the first attachment detachment portion 1501, and a flexible portion model NM3 that models the extracorporeal flexible portion 140 and the internal flexible portion 119, and a curved portion model NM4 that models the curved portion 112.

The flexible part model NM3 is a model that can estimate the displacement (distal displacement) and tension on the distal side of the bending wire 160 from the displacement (proximal displacement) on the proximal side of the bending wire 160 that is inserted. The flexible portion model NM3 includes a friction coefficient μ and a stiffness EA as control parameters.

The control parameter of the flexible portion model NM3 needs to be periodically calibrated and updated because the bending wire 160, the curved portion 112, and the flexible portion (the insertion portion 110 and the extracorporeal flexible portion 140) deteriorate over time. Possible deterioration due to change is, for example, deformation or increase in friction of the curved piece 115, increase in friction due to peeling of the coating on the flexible portion (the insertion portion 110 and the extracorporeal flexible portion 140), elongation of the bending wire 160, shrinkage of the sheath, and the like.

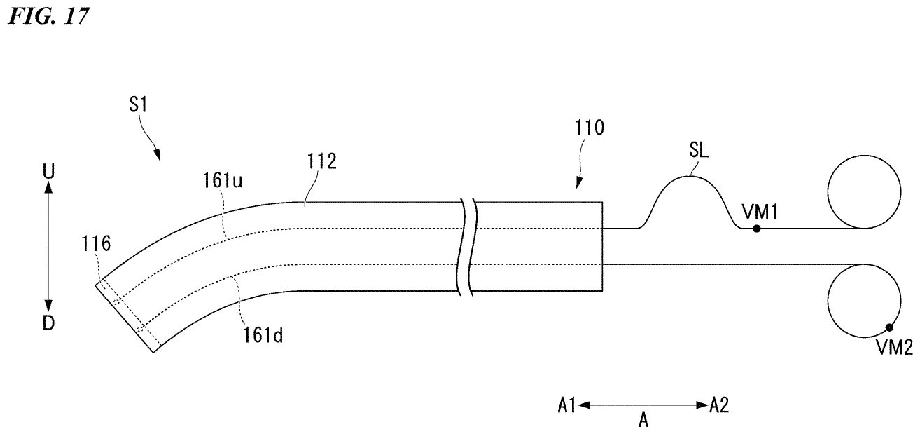

FIGS. 17 to 21 are diagrams showing a pair of bending wires 160 inserted through the bending insertion portion 110. A pair of bending wires (an upper bending wire 161u and a lower bending wire 161d) for bending the curved portion 112 in the UD direction will be described below. A virtual marker VM1 and a virtual marker VM2 are virtual markers that indicate the position of the internal distance from the top of a pair of bending wires (the upper bending wire 161u and the lower bending wire 161d). A pair of bending wires 160 (a left bending wire 161l and a right bending wire 161r) for bending the curved portion 112 in the LR direction have the same structure, so illustration and description are omitted.

A pair of bending wires 160 shown in FIG. 17 is in a state (also referred to as first state S1) in Which the lower bending wire 161d bends the curved portion 112 most in the D direction.

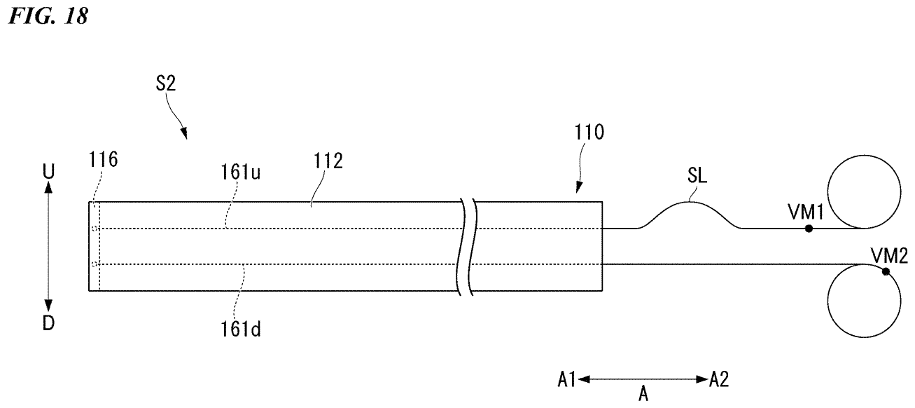

A pair of bending wires 160 shown in FIG. 18 is in a state where the lower bending wire 161d starts bending the curved portion 112 in the D direction (also referred to as second state S2).

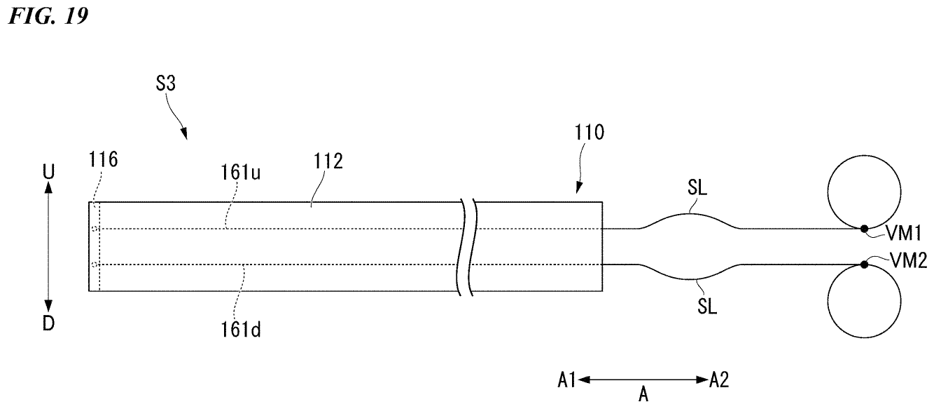

A pair of bending wires 160 shown in FIG. 19 is in a state (also referred to as third state S3) in which the pair of bending wires 160 make the curved portion 112 in a straight shape without bending.

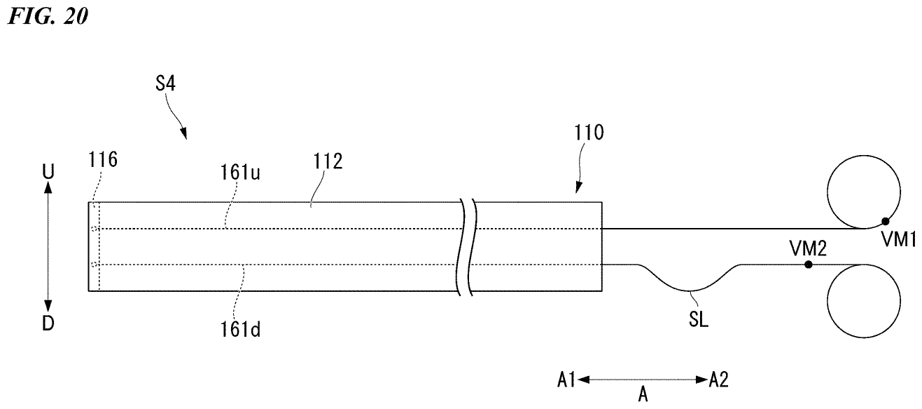

A pair of bending wires 160 shown in FIG. 20 is in a state where the upper bending wire 161u starts bending the curved portion 112 in the U direction (also referred to as fourth state S4).

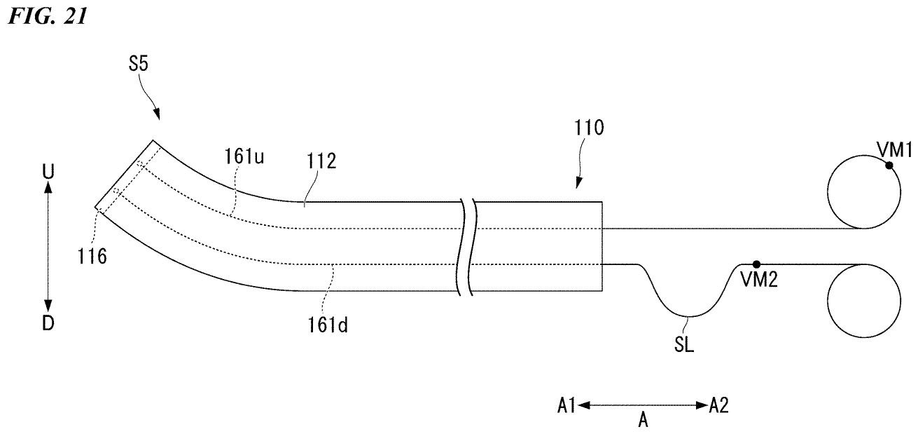

A pair of bending wires 160 shown in FIG. 21 is in a state (also referred to as fifth state S5) in which the upper bending wire 161u bends the curved portion 112 most in the U direction.

The pair of bending wires 160 changes in path length according to the bending of the flexible portion (insertion portion 110 and extracorporeal flexible portion 140). Therefore, the pair of bending wires 160 has a surplus length capable of absorbing the change in path length, and has “slack SL” in the third state S3 shown in FIG. 19.

### [Operation of the Electric Endoscope System 1000C]

Next, the operation of an electric endoscope system 1000C of this embodiment will be described. Specifically, operations related to calibration for updating the flexible part model NM3 will be described.

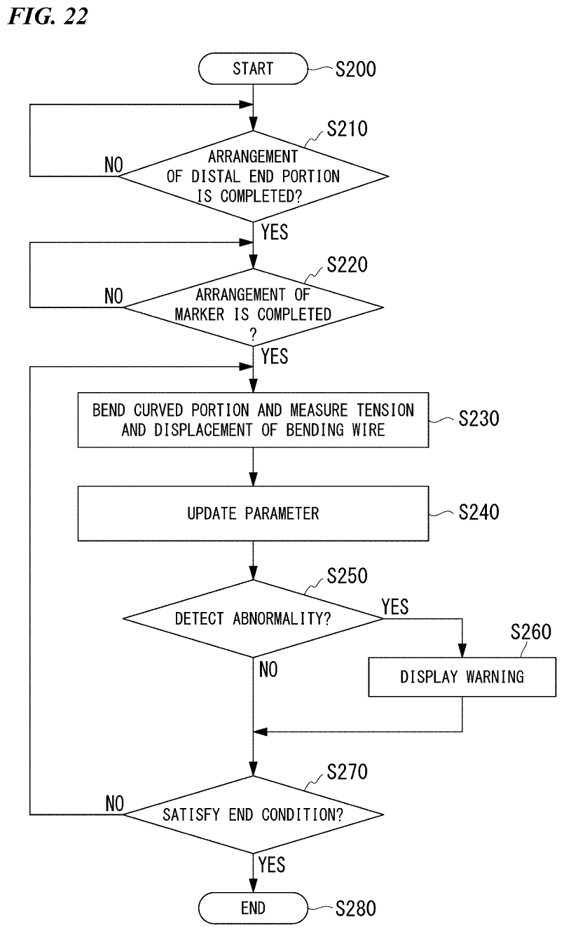

Hereafter, a description will be made according to the control flowchart of the main controller 560 of the control device 600 shown in FIG. 22. When the user starts the “calibration program” in the control device 600, the main controller 560 starts the control flow shown in FIG. 22 (step S200). Next, the main controller 560 (mainly the processor 561) executes step S210.

### <Step S210: Arrangement Step/Shape Estimation Step>

In step S210, the main controller 560 displays on the display device 900 a GUI image that instructs the user to hang the endoscope 100 on the hanger 710 and hang the distal end portion 180 including the curved portion 112 of the endoscope 100 from the hanger 710.

The main controller 560 can estimate the shape of the flexible portion (the insertion portion 110 and the extracorporeal flexible portion 140) installed on the hanger 710. The main controller 560 can, in particular, accurately estimate the shape of the insertion portion 110 from the lifted connecting portion 120 to the distal end portion 111. If the height at which the hanger 710 is installed is known, the main controller 560 can also accurately estimate the shape of the extracorporeal flexible portion 140. Next, the main controller 560 executes step S220.

### <Step S220: Marker Arrangement Step>

The main controller 560 displays on the display device 900 a GUI image instructing the user to arrange the marker M in step S220. The user arranges the marker M according to the instructions displayed on the GUI image, as in the second embodiment. Since the marker M is not essential, the main controller 560 may omit step S220 (marker arrangement step). Next, the main controller 560 executes step S230.

### <Step S230: Measurement Step>

In step S230, the main controller 560 drives the curved portion 112 in the UD direction to measure the tension and displacement of the pair of bending wires 160 (the upper bending wire 161u and the lower bending wire 161d). Specifically, the drive controller 260 (mainly a processor) bends the curved portion 112 directed in the D direction in the U direction by the upward bending wire 161u, as shown in FIGS. 17 to 21. Note that the drive controller 260 bends the curved portion 112 facing the U direction also in the D direction by the lower bending wire 161d, but as the control is the same, the explanation is omitted.

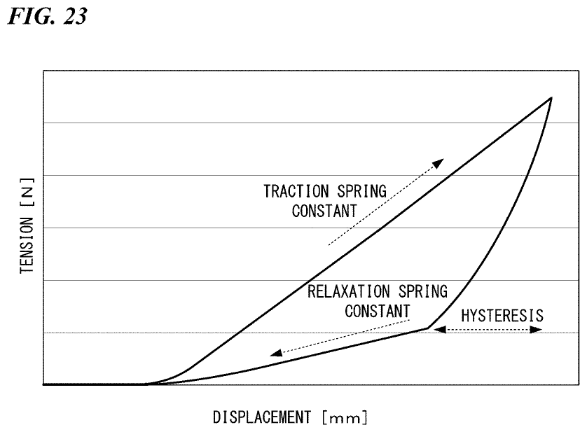

FIG. 23 is a graph showing the relationship between the measured tension and displacement of the upward bending wire 161u.

The main controller 560 acquires the tension (proximal end tension) of the upper bending wire 161u from the tension sensor, and acquires the displacement (proximal end displacement) of the upper bending wire 161u from the encoder of the wire drive portion 250. The main controller 560 estimates the traction spring constant, the hysteresis, and the relaxation spring constant based on the measurement results.

The pulling spring constant is a spring constant that can be calculated from the relationship between the tension and displacement of the upward bending wire 161u from the fourth state to the fifth state.

The hysteresis is the relaxation amount of the upward bending wire 161u until the curved portion 112 begins to bend in the D direction (dead zone) when the upward bending wire 161u is relaxed in the fifth state. The main controller 560 estimates the hysteresis based on the boundary point where the slope changes significantly in the tension-displacement graph of the upper bending wire 161u. When the marker M is arranged, the main controller 560 can more accurately estimate the hysteresis by detecting that the curved portion 112 actually starts bending in the D direction.

The relaxed spring constant is a spring constant that can be calculated from the relationship between tension and displacement of the upward bending wire 161u from the state where the curved portion 112 starts bending in the D direction to the third state.

After estimating the traction spring constant, the hysteresis, and the relaxation spring constant, the main controller 560 then executes step S240.

### <Step S240: Adjustment Step>

In step S240, the main controller 560 controls the control parameters (friction coefficient μ and stiffness EA), The main controller 560 then executes step S250.

### <Step S250: Abnormality Determination>

In step S250, the main controller 560 confirms whether the control parameters (friction coefficient μ and stiffness EA) of the flexible portion model NM3 are values within the normal range. When the control parameter of the flexible part model NM3 is not a numerical value within the normal range but is abnormal, the main controller 560 executes step S260 to display a GUI image presenting the abnormality to the user on the display device 900. When the control parameter of flexible part model NM3 is a numerical value within the normal range, main controller 560 executes step S270.

### <Step S270: End Determination Step>

The main controller 560 determines the end of calibration in step S270. For example, When the calibration is performed in each of the UD direction and the LR direction, the main controller 560 determines that the calibration has been completed, executes step S280, and ends the control flow shown in FIG. 22. When the main controller 560 determines that the calibration has not been completed, such as when an abnormality occurs, the main controller 560 executes step S230 again.

According to the electric endoscope system 1000C according to the present embodiment, in a state in which the shape of the flexible portion (insertion portion 110 and extracorporeal flexible portion 140) can be easily estimated, such as a state in which the endoscope 100 is hung on the hanger 710, the reference model NM can be easily calibrated without using a special jig for calibration. Therefore, even if the operating room or treatment room in which the electric endoscope system 1000C is installed is small, the control device 600 (mainly the main controller 560) can easily perform calibration. The main controller 560 can precisely drive the endoscope 100 using the updated reference model NM.

As described above, the third embodiment of the present disclosure has been described in detail with reference to the drawing, but the specific configuration is not limited to this embodiment, and design changes etc. are included within the scope of the present disclosure. Also, the constituent elements shown in the above-described embodiment and modifications can be combined as appropriate.

### Modification 3-1

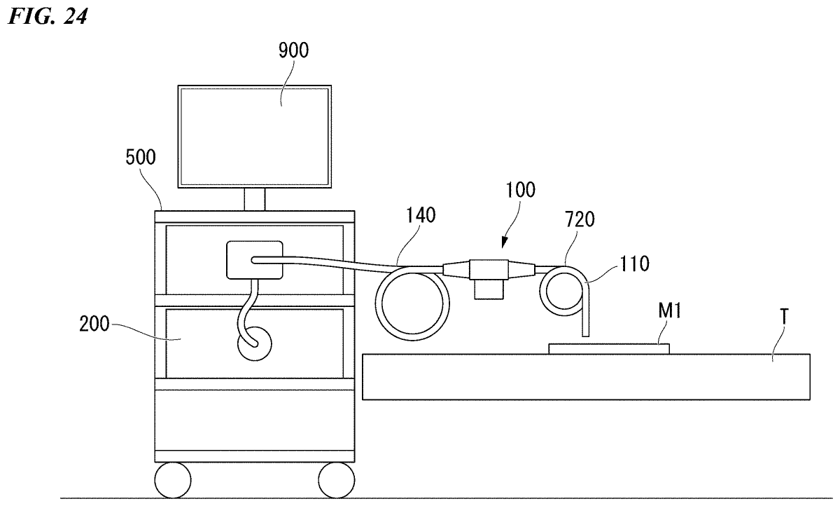

FIG. 24 is a diagram showing another aspect of step S210 (arrangement step).

The flexible portion (insertion portion 110 and extracorporeal flexible portion 140) of the endoscope 100 may be housed in a dedicated jig 720 in step S210. The jig 720 is positioned to accommodate the flexible portion. Therefore, the main controller 560 can accurately estimate the shape of the flexible portion.

### Modification 3-2

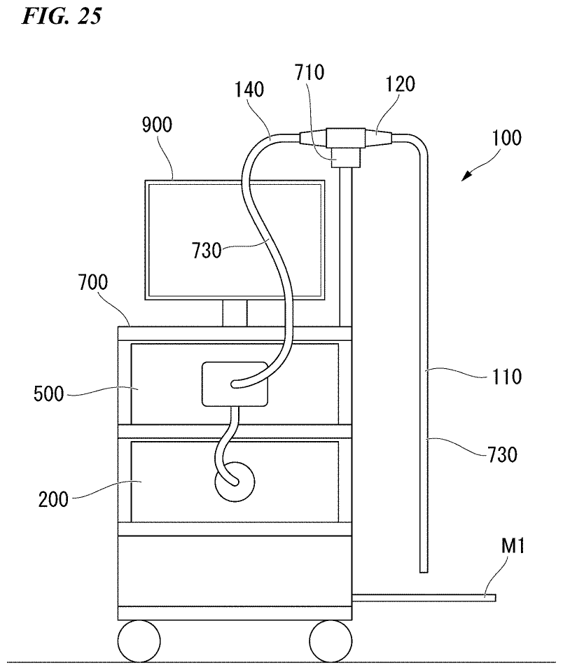

FIG. 25 is a diagram showing another aspect of step S210 (arrangement step).

The flexible portion (insertion portion 110 and extracorporeal flexible portion 140) of the endoscope 100 may have a sensor 730 capable of measuring the shape, as shown in FIG. 25. The sensor 730 is, for example, a magnetic coil whose shape can be measured using a magnetic field. The main controller 560 can accurately estimate the shape of the flexible portion.

### Modification 3-3

Although the calibration of the endoscope 100 is mainly performed by the main controller 560 in the above embodiment, it may be performed by the drive controller 260 as well.

### Fourth Embodiment

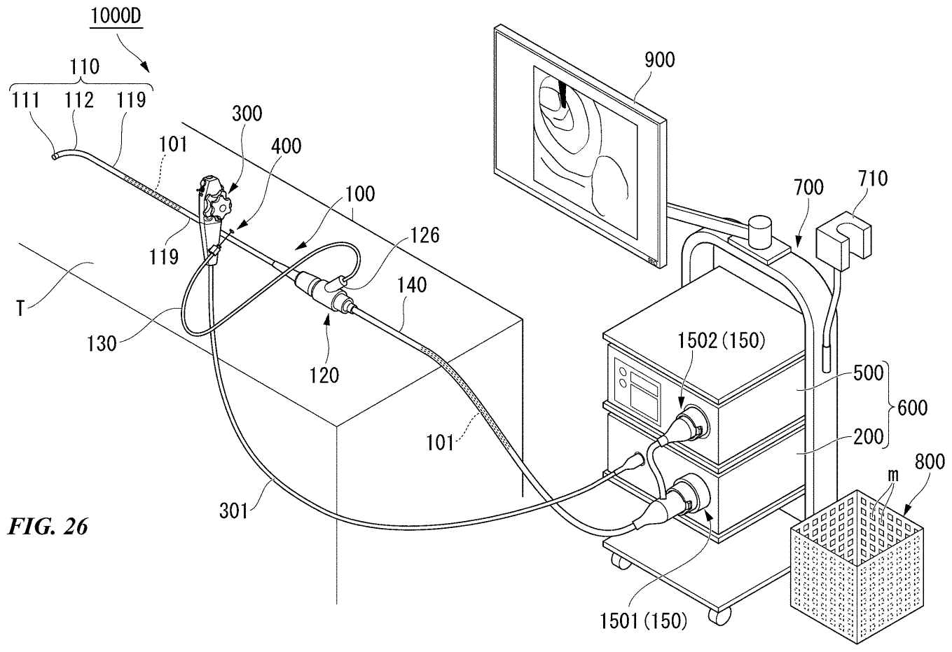

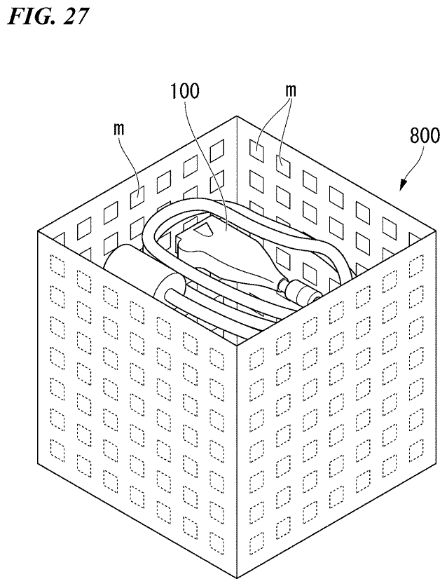

An electric endoscope system 1000D according to a fourth embodiment of the present disclosure will be described with reference to FIGS. 26 to 27. In the following description, the same reference numerals are given to the same configurations as those already described, and redundant descriptions will be omitted.

### [Electric Endoscope System 1000D]

FIG. 26 is an overall view of the electric endoscope system 1000D according to this embodiment.

The electric endoscope system 1000D includes an endoscope 100, a driving device 200, an operation device 300, a treatment tool 400, an image control device 500, a display device 900, a storage rack 700, and a packing box 800.

FIG. 27 is a diagram showing the packing box 800.

The packing box 800 is a box for packing the endoscope 100, which is used for surgery and of which cleanliness for use in surgery is guaranteed, and disposable items (disposables). The packing box 800 is discarded after each operation (case). The packing box 800 is formed in a substantially box shape. The packing box 800 has a known marker pattern m on its inner surface, like the marker M2 of the second embodiment.

The user can calibrate the endoscope 100 by measuring the shape information of the curved portion 112 using the packing box 800 like the marker M2 of the second embodiment. Also, the user can use the packing box 800 like the marker M1 of the third embodiment to determine the relationship between the tension and displacement of the bending wire 160 to perform calibration.

According to the electric endoscope system 1000D of the present embodiment, the endoscope 100 can be calibrated using the packaging box 800 that is discarded after each operation case), thus ensuring cleanliness and saving space.

As described above, the fourth embodiment of the present disclosure has been described in detail with reference to the drawings, but the specific configuration is not limited to this embodiment, and design changes etc. are included within the scope of the present disclosure. Also, the constituent elements shown in the above-described embodiment and modifications can be combined as appropriate.

### Modification 4-1

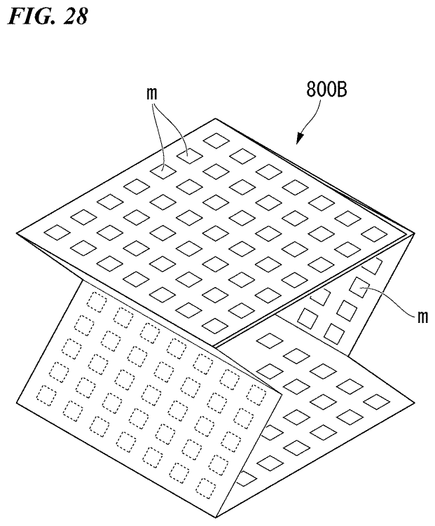

FIG. 28 is a diagram showing a packaging box 800B as a modified example of the packaging box 800.

The packing box 800B is a foldable box. The packaging box 800B, like the packaging box 800, has a known marker pattern m on its inner surface. By assembling the packaging box 800B, a large marker pattern m can be used without increasing the size of the packaging box 800B itself.

### Modification 4-2

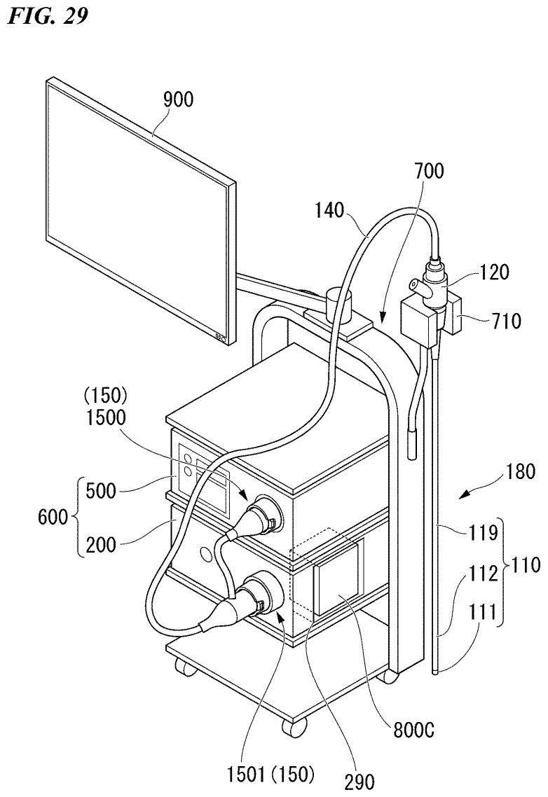

FIG. 29 is a diagram showing a packaging box 800C as a modification of the packaging box 800.

The packaging box 800C has the same configuration as the packaging box 800, and is detachable from the driving device 200. The packing box 800C can be stored in a recess 290 formed in the driving device 200. Since the driving device 200 can be stored in the packing box 800C, space saving can be achieved. Note that the packing box 800C may be detachable from the video control device 500.

Each of the above embodiments may be realized by recording the program on a computer-readable recording medium, loading the program recorded on the recording medium into a computer system, and executing the program. The “computer system” includes hardware such as an OS and peripheral devices. Further, the “computer-readable recording medium” refers to a portable medium such as a flexible disk, a magneto-optical disk, a ROM, or a CD-ROM, or a storage device such as a hard disk built in a computer system. Further, the “computer-readable recording medium” may include a medium that dynamically holds a program for a short period of time, such as a communication line when a program is transmitted via a network such as the Internet or a communication line such as a telephone line, and a medium that holds a program for a certain period of time, such as a volatile memory inside a computer system that serves as a server or client in that case. Further, the above-mentioned program may be a program for realizing some of the above-mentioned functions, and may be a program for realizing the above-mentioned functions in combination with a program already recorded in the computer system.

What is claimed is:

1. A method for calibrating an endoscope, the endoscope including a curved portion that can be driven to bend and a flexible portion through which a bending wire that bends the curved portion is inserted, the method comprising:
arranging the curved portion and the flexible portion of the endoscope so that they have a predetermined shape;
bending the curved portion;
measuring first shape information, which is information about a shape of the curved portion; and
updating a control parameter for the endoscope to drive the curved portion based on the measured first shape information.

2. The method for calibrating the endoscope according to claim 1, wherein,
a distal end portion of the endoscope, including the curved portion, is suspended and arranged so that the distal end portion is straight in a vertical direction when arranging the curved portion and the flexible portion of the endoscope.

3. The method for calibrating the endoscope according to claim 1, wherein
the control parameter is a parameter used when calculating a control amount for driving the curved portion from an operation input to the curved portion.

4. The method for calibrating the endoscope according to claim 2, further comprising
placing a marker having a marker pattern that can identify relative position information below the distal end portion in the vertical direction,
wherein, the first shape information of the curved portion is measured based on an image of the marker pattern of the marker when measuring the first shape information.

5. The method for calibrating the endoscope according to claim 1, wherein
measuring the first shape information includes measuring, as the first shape information, a displacement and tension of the bending wire when bending the curved portion, and
updating the control parameter includes updating the control parameter for the endoscope to drive the curved portion based on the measured first shape information.

6. The method for calibrating the endoscope according to claim 5, further comprising
estimating second shape information, which is information about a shape of the flexible portion, and
updating the control parameter includes updating a control parameter for the endoscope to drive the curved portion based on the estimated second shape information and the first shape information. The method for calibrating the endoscope according to claim 6, wherein,
the flexible portion is suspended and arranged such that al least a part of the flexible portion is in a straight line along a vertical direction when arranging the curved portion and the flexible portion of the endoscope.

8. The method for calibrating the endoscope according to claim 7, wherein
the control parameter includes a friction coefficient or a coefficient related to stiffness of the flexible portion.

9. An endoscope system, comprising:
an endoscope including a curved portion that can be driven to bend, and a flexible portion through which a bending wire that bends the curved portion is inserted; and
a control device including a processor that controls driving of the curved portion,
wherein the processor is configured perform:
bending the curved portion;
measuring first shape information, which is information about a shape of the curved portion; and
updating a control parameter for the endoscope to drive the curved portion based on the measured first shape information.

10. The endoscope system according to claim 9, wherein the processor is configured to perform, before bending the curved portion confirming that a distal end portion including the curved portion of the endoscope has been suspended and arranged

11. The endoscope system according to claim 10, wherein the processor is configured to perform, before bending the curved portion, confirming that a marker having a marker pattern capable of identifying relative position information has been arranged below the distal end portion in a vertical direction.

12. The endoscope system according to claim 11, wherein the processor is configured to instruct a user to rearrange the marker when the endoscope cannot image the marker pattern both before and after bending the curved portion when bending the curved portion.

13. The endoscope system according to claim 12, wherein the marker is formed in a sheet shape.

14. The endoscope system according to claim 13, wherein the marker has a plurality of surfaces having the marker pattern.

15. The endoscope system according to claim 14, further comprising
a driving device configured to generate power for driving the endoscope according to a signal from the control device,
wherein the marker is formed in a box shape haying a surface with the marker pattern, and is detachable from the driving device or the control device.

16. The endoscope system according to claim 9, wherein
measuring a displacement and tension of the bending wire when bending the curved portion as the first shape information, and
updating the control parameter for the endoscope to drive the curved portion based on the measured first shape information.

17. The endoscope system according to claim 16, wherein
the processor further performs estimating second shape information, which is information related to a shape of the flexible portion, and
updating the control parameter includes updating a control parameter for the endoscope to drive the curved portion based on the estimated second shape information and the first shape information.

18. The endoscope system according to claim 10, wherein, the flexible portion is suspended and arranged such that at least a part of the flexible portion is in a straight line along a vertical direction when confirming that a distal end portion including the curved portion of the endoscope has been suspended and arranged.

19. The endoscope system according to claim 18, the endoscope is arranged on a jig in which a position where the flexible portion is accommodated is determined when confirming that a distal end portion including the curved portion of the endoscope has been suspended and arranged.

20. The endoscope system according to claim 19, wherein the control parameter includes a friction coefficient or a coefficient related to stiffness of the flexible portions.
